(19) United States
(12) Patent Application Publication
(10) Pub. No.: US 2020/0087680 A1
(43) Pub. Date: Mar. 19, 2020

(54) Conditional Living Sensors

(71) Applicant: NantBio, Inc., Culver City, CA (US)

(72) Inventors: Kayvan Niazi, Encino, CA (US); Wael Tadros, Los Angeles, CA (US); Clifford Anders Olson, Long Beach, CA (US); Nicholas James Witchey, Laguna Hills, CA (US)

(21) Appl. No.: 16/569,582

(22) Filed: Sep. 12, 2019

Related U.S. Application Data
(60) Provisional application No. 62/730,981, filed on Sep. 13, 2018.

(51) Int. Cl. C12N 15/85; A61K 9/00; G01N 33/50; A61K 48/00

(52) U.S. Cl. CPC C12N 15/85; A61K 9/0014; A61K 9/0053; A61K 48/0083; A61K 48/0075; A61K 48/0058; G01N 33/5091

(57) ABSTRACT

The present disclosure relates to recombinant sensor cells comprising an AND gate such that an expressible sequence (e.g., a reporter gene) is expressed after the occurrence of two separate triggering events. Nucleic acids, kits, and methods for making and using the recombinant sensor cells are also disclosed herein.

### CROSS-REFERENCE TO RELATED APPLICATIONS

This application claims priority to U.S. Provisional Application No. 62/730,981 filed Sep. 13, 2018, the disclosures of which are incorporated herein by reference in its entirety.

### FIELD OF THE INVENTION

The field of the invention is recombinant sensor cells for sensing one or more conditions, e.g., in the tumor microenvironment, such that an expressible sequence (e.g., a reporter gene) is expressed after the conditions are sensed by the cell.

### BACKGROUND

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

With the advent of personalized therapy for various cancers, the importance of identifying cancers that are likely (or unlikely) to be treated by a particular therapy has increased. For example, several FDA-approved cancer treatments use genetic analysis of a tumor to identify patients who should receive the treatment. Other information about a tumor can also be important to determining the course of treatment, including location of the edges of a tumor and/or metastases, phenotype of the tumor, response of the tumor to a particular treatment, and the like.

Thus, there remains a need for compositions and methods to provide relevant information about a tumor to a clinician.

### SUMMARY OF THE INVENTION

The instant technology generally relates to recombinant sensor cells comprising an AND gate such that an expressible sequence (e.g., a reporter gene) is expressed after the occurrence of two separate triggering events. In some embodiments, the recombinant sensor cells comprise a logic gate or circuit (e.g., NOR, OR, XOR, NAND, XNOR, NOT, multiplexer, encoders, decoders, combinations of gates, etc.) where the expression of one or more expressible sequences is regulated by the occurrence of two or more separate triggering events. Nucleic acids, kits, and methods for making and using the recombinant sensor cells are also disclosed.

Although Weinberg, et al. (Nature Biotechnology 35, pages 453-462 (2017), incorporated herein by reference in its entirety) provides a design for genetic circuits having multiple inputs and outputs in mammalian cells, their design relies on recombinases and is not a reversible system. That is, once the cells containing the genetic circuits are exposed to the recombinase(s), the relevant DNA pieces are excised or inverted, and this cannot be undone.

In contrast, the recombinant sensor cells described herein allow for the reporter to be turned on or off, depending on the microenvironment experienced by the cell at a given time. Thus, the technology described herein provides a dynamic reporter system utilizing genetic circuits to monitor the reporter cell's microenvironment and provide real-time feedback via reporter expression. Further, the cells described herein can be modified to express therapeutic molecules, instead of or in addition to a reporter, in response to signals from the microenvironment, and optionally to turn off expression of those therapeutic molecules when the microenvironment changes (e.g., when the cell is no longer in the presence of tumor cells).

The recombinant sensor cells comprise genetically modified cells comprising logic cassettes, such that the presence or absence of at least two signals (e.g., from one or more triggering events) results in activation or repression of expression from a reporter gene. In this way, the recombinant sensor cells identify a set of conditions, for example in a cellular microenvironment in a patient, and report on the presence (or absence) of those conditions. In some embodiments, the sensor cells sense one or more conditions in an environment, such as a tumor microenvironment.

A particular condition, referred to herein as a “triggering event,” results in a signal (e.g., presence or binding of a ligand, lack of binding, etc.). The sensor cell expresses at least two recombinant proteins, each of which senses a distinct triggering event by binding the signal (e.g., ligand), and is thereby activated by the signal. The activated recombinant proteins activate expression of an expressible sequence, such as a reporter, when both recombinant proteins are present and activated. If one or no recombinant proteins is activated, the expressible sequence is not expressed. This system allows expression of, for example, a reporter protein only when the sensor cell is in the presence of a particular type of cellular (e.g., tumor, specific tissue, specific organ, bacteria, etc.) microenvironment.

Alternatively, the sensor cell may express the expressible sequence, e.g. reporter, and this expression is repressed when both recombinant proteins are present and activated. If one or no recombinant proteins is activated, the expressible sequence is expressed. This system allows expression of, for example, a reporter protein only when the sensor cell is not in the presence of a particular type of cellular microenvironment.

In some embodiments, the recombinant sensor cell comprises:
(i) a first sensor cassette comprising a first nucleic acid sequence encoding a first protein having a first ligand binding portion and a first activator portion, wherein the first ligand binding portion is capable of binding a first ligand from a first triggering event; and
(ii) a second sensor cassette comprising a second nucleic acid sequence encoding a second protein having a second ligand binding portion and a second activator portion, wherein the second ligand binding portion is capable of binding a second ligand from a second triggering event;
(iii) a first logic cassette comprising a third nucleic acid sequence comprising a promoter sequence operably linked to an expressible sequence, wherein expression from the promoter sequence is activated by the first activator portion and the second activator portion, such that the expressible sequence is expressed only after the first and second triggering events occur.

In some embodiments, the recombinant sensor cell comprises:
(i) a first sensor cassette comprising first nucleic acid sequence comprising a first signal-inducible promoter operably linked to a first expressible sequence encoding a first activator, wherein expression of the first activator is induced by a first signal from a first triggering event;
(ii) a second sensor cassette comprising a second nucleic acid sequence comprising a second signal-inducible promoter operably linked to a second expressible sequence encoding a second activator, wherein expression of the second activator is induced by a second signal from a second triggering event; and
(iii) a first logic cassette comprising a third nucleic acid sequence comprising a promoter sequence operably linked to a third expressible sequence, wherein the promoter is responsive to the first activator and the second activator;
wherein the first activator and the second activator activate expression from the promoter sequence only when both the first activator and the second activator are expressed.

Preferably, each of the first, second and third nucleic acid sequences are recombinant nucleic acid sequences, i.e., they are not endogenous to the cell.

In some embodiments, the first activator comprises a chromatin remodeler, a histone acetyltransferase, a histone deacetylase, a kinase, a methylase, a transcription factor, or a transcription co-factor. In some embodiments, the second activator comprises a chromatin remodeler, a histone acetyltransferase, a histone deacetylase, a kinase, a methylase, a transcription factor, or a transcription co-factor.

In some embodiments, the recombinant sensor cell comprises:
(i) a first sensor cassette comprising first nucleic acid sequence comprising a first signal-inducible promoter operably linked to a first expressible sequence encoding a first protein comprising a DNA binding portion and a first linker portion, wherein expression of the first protein is induced by a first signal from a first triggering event;
(ii) a second sensor cassette comprising a second nucleic acid sequence comprising a second signal-inducible promoter operably linked to a second expressible sequence encoding a second protein comprising an activator portion and a second linker portion, wherein expression of the second protein is induced by a second signal from a second triggering event, wherein interaction between the first linker portion and the second linker portion forms a functional activator protein; and
(iii) a first logic cassette comprising a third nucleic acid sequence comprising a promoter sequence operably linked to a third expressible sequence, wherein the promoter is responsive to the functional activator protein;
wherein the first protein and the second protein activate expression from the promoter sequence only when both the first protein and the second protein are expressed, such that the expressible sequence is expressed only when both the first signal and the second signal are present.

In some embodiments, the activator portion comprises a chromatin remodeler, a histone acetyltransferase, a histone deacetylase, a kinase, a methylase, a transcription factor, or a transcription co-factor.

In some embodiments, the recombinant sensor cell comprises:
(i) a first sensor cassette comprising a first nucleic acid sequence encoding a protein having a first receptor moiety and a first activating moiety, wherein the first receptor moiety interacts with a first signal from the first triggering event;
(ii) a second sensor cassette comprising a second nucleic acid sequence encoding a protein having a second receptor moiety and a second activating moiety, wherein the second receptor moiety interacts with a second signal from the second triggering event; and
(iii) a first logic cassette comprising a third nucleic acid sequence comprising a promoter operably linked to an expressible sequence, wherein expression from the promoter is activated by the first activating moiety and the second activating moiety, such that the expressible sequence is expressed only after the first and second triggering events occur.

In some embodiments, the first signal results in ligand binding, phosphorylation, ubiquitination, hydrolysis, nitration, sulfhydration, acetylation, lipid modification, methylation, glycosylation, propionylation, butyrylation, succinylation, malonylation, palmitoylation, and/or crotonylation of the first receptor moiety.

In some embodiments, the second signal results in ligand binding, phosphorylation, ubiquitination, hydrolysis, nitration, sulfhydration, acetylation, lipid modification, methylation, glycosylation, propionylation, butyrylation, succinylation, malonylation, palmitoylation, and/or crotonylation of the second receptor moiety.

In some embodiments, the first triggering event and the second triggering event are independently selected from cell density, pH, hypoxia, radio signal, MRI, heat, presence of a molecule of interest, or concentration of a molecule of interest. In some embodiments, the first triggering event and/or the second triggering event is present in a tumor cell microenvironment.

In some embodiments, the molecule of interest is a cytokine, a chemokine, a metabolite, an exosome, an enzyme, a sugar, an intracellular component, a soluble checkpoint inhibitor, a signaling factor, a virus, a yeast cell, or a bacterial cell.

In some embodiments, the expressible sequence encodes a reporter. In some embodiments, the reporter is selected from a fluorescent protein, a cell surface marker, a detectable RNA molecule, a detectable DNA molecule, a luciferase, or a reporter enzyme.

In some embodiments, the expressible sequence encodes a therapeutic molecule, a cytotoxic pathway molecule, a pro-apoptotic protein, an immunostimulator, or an immunorepressor.

In some embodiments, the recombinant sensor cell is an immune cell, a stem cell, a bacterial cell, or a parasite. In some embodiments, the immune cell is an immunocompetent cell. In some embodiments, the immune cell is a natural killer (NK) cell, a B cell, or a T cell. In some embodiments, the cell is derived from a patient. In some embodiments, the NK cell is a NK-92 cell.

In some embodiments, the bacterial cell is Escherichia coli. In some embodiments, the bacterial cell does not trigger the endotoxic response in mammalian cells. In some embodiments, the bacterial cell is a ClearColi® cell (Lucigen®, Madison, Wis.).

In some embodiments, the parasite is a nematode, a spirochete, or a fungus.

In some embodiments, the recombinant sensor cell is an enucleated cell that is capable of transcription and translation. In some embodiments, the recombinant sensor cell is an exosome that is capable of transcription and translation.

In some embodiments, the nucleic acids (e.g., (i), (ii), and/or (iii)) are transiently transfected into the cell. In some embodiments, the nucleic acids (e.g., (i), (ii), and/or (iii)) are stably transfected into the cell.

In some embodiments, the nucleic acids (e.g., (i), (ii), and/or (iii)) are present on one or more vectors in the cell.

In some embodiments, the recombinant sensor cell further comprises at least one additional logic cassette comprising a fourth nucleic acid sequence comprising a second promoter sequence operably linked to a second expressible sequence, wherein expression from the second promoter sequence is activated by the first activator portion and the second activator portion, such that the second expressible sequence is expressed only after the first and second triggering events occur.

In some embodiments, the second expressible sequence encodes a reporter. In some embodiments, the second expressible sequence encodes a therapeutic molecule, a cytotoxic pathway molecule, a pro-apoptotic protein, an immunostimulator, or an immunorepressor.

In some embodiments, the expressible sequence and the second expressible sequence are different sequences. Preferably, the protein or mRNA encoded by the second expressible sequence is different from a protein or mRNA encoded by the expressible sequence.

In some embodiments is provided a cell line comprising a plurality of the recombinant sensor cell described herein.

In some embodiments is provided a topical composition comprising a recombinant sensor cell as described herein.

In some embodiments is provided a composition comprising a recombinant sensor cell as described herein which is formulated for oral, nasal, vaginal, or anal delivery.

In some embodiments is provided a method for producing a recombinant therapeutic cell as described herein, the method comprising introducing the first logic cassette, first sensor cassette, and second sensor cassette into the cell.

In some embodiments is provided a method of detecting at least two triggering events in a tumor microenvironment, the method comprising contacting the tumor microenvironment with a recombinant sensor cell as described herein, and determining the presence or absence of an RNA or polypeptide expressed from the expressible sequence.

In some embodiments is provided a method of treating a tumor, the method comprising contacting the tumor microenvironment with a recombinant sensor cell as described herein, wherein the expressible sequence encodes a molecule that treats the tumor.

In some embodiments, the molecule that treats the tumor is an immune stimulating cytokine, a chemokine, a pro-apoptotic protein, an antisense RNA, a chimeric antigen receptor, or a therapeutic antibody.

In some embodiments, the tumor is in a subject and the recombinant sensor cell is derived from the subject.

In some embodiments is provided a method of detecting at least two triggering events in a biological sample, the method comprising contacting the biological sample with a recombinant sensor cell as described herein, and determining the presence or absence of an RNA or polypeptide expressed from the expressible sequence.

In some embodiments, the biological sample is from a subject having or suspected of having cancer. In some embodiments, the biological sample comprises blood, urine, a biopsy, saliva, hair, skin, or feces. In some embodiments, the biological sample comprises cell free DNA and/or cell free RNA.

In some embodiments is provided a recombinant vector comprising one or more of:
(i) a first sensor cassette comprising a first nucleic acid sequence encoding a protein having a first receptor moiety and a first activating moiety, wherein the first receptor moiety binds a first signal from the first triggering event;
(ii) a second sensor cassette comprising a second nucleic acid sequence encoding a protein having a second receptor moiety and a second activating moiety, wherein the second receptor moiety binds a second signal from the second triggering event; and
(iii) a first logic cassette comprising a third nucleic acid sequence comprising a promoter operably linked to an expressible sequence, wherein expression from the promoter is activated by the first activating moiety and the second activating moiety, such that the expressible sequence is expressed only after the first and second triggering events occur.

In some embodiments is provided a kit comprising:
(i) a first sensor cassette comprising a first nucleic acid sequence encoding a first protein having a first ligand binding portion and a first activator portion, wherein the first ligand binding portion is capable of binding a first signal from the first triggering event; and
(ii) a second sensor cassette comprising a second nucleic acid sequence encoding a second protein having a second ligand binding portion and a second activator portion, wherein the second ligand binding portion is capable of binding a second signal from the second triggering event;
(iii) a first logic cassette comprising a third nucleic acid sequence comprising a promoter sequence operably linked to an expressible sequence, wherein expression from the promoter sequence is activated by the first activator portion and the second activator portion, such that the expressible sequence is expressed only after the first and second triggering events occur;
such that when both the first signal and the second signal are present the expressible sequence is expressed.

In some embodiments is provided a kit comprising:
(i) a first sensor cassette comprising first nucleic acid sequence comprising a first signal-inducible promoter operably linked to a first expressible sequence encoding a first activator, wherein expression of the first activator agent is induced by a first signal from the first triggering event;
(ii) a second sensor cassette comprising a second nucleic acid sequence comprising a second signal-inducible promoter operably linked to a second expressible sequence encoding a second activator, wherein expression of the second activator agent is induced by a second signal from the second triggering event; and
(iii) a first logic cassette comprising a third nucleic acid sequence comprising a promoter sequence operably linked to a third expressible sequence, wherein the promoter is responsive to the first activator and the second activator;
wherein the first activator and the second activator activate expression from the promoter sequence only when both the first activator and the second activator are present, such that the expressible sequence is expressed only when both the first signal and the second signal are present.

In some embodiments is provided a kit comprising:
(i) a first sensor cassette comprising a first nucleic acid sequence encoding a protein having a first receptor moiety and a first activating moiety, wherein the first receptor moiety binds a first signal from the first triggering event;
(ii) a second sensor cassette comprising a second nucleic acid sequence encoding a protein having a second receptor moiety and a second activating moiety, wherein the second receptor moiety binds a second signal from the second triggering event; and
(iii) a first logic cassette comprising a third nucleic acid sequence comprising a promoter operably linked to an expressible sequence, wherein expression from the promoter is activated by the first activating moiety and the second activating moiety, such that the expressible sequence is expressed only after the first and second triggering events occur.

### BRIEF DESCRIPTION OF THE FIGURES

FIGS. 1 and 2 are schematics of exemplary recombinant sensor cells as described herein.

### DETAILED DESCRIPTION

After reading this description it will become apparent to one skilled in the art how to implement the invention in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

### Definitions

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, the phrase “at least one of A and B” is intended to refer to ‘A’ and/or ‘B’, regardless of the nature of ‘A’ and ‘B’. For example, in some embodiments, ‘A’ may be single distinct species, while in other embodiments ‘A’ may represent a single species within a genus that is denoted ‘A’. Likewise, in some embodiments, ‘B’ may be single distinct species, while in other embodiments ‘B’ may represent a single species within a genus that is denoted ‘B’.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization. The term “cell” also may refer to an exosome or enucleated cell that contains sufficient intracellular machinery to carry out transcription and translation.

“Contacting” is used in accordance with its plain ordinary meaning and refers to the process of allowing at least two distinct species (e.g. chemical compounds including biomolecules or cells) to become sufficiently proximal to react, interact or physically touch.

The term “expression” includes any step involved in the production of the polypeptide including, but not limited to, transcription, post-transcriptional modification, translation, post-translational modification, and secretion.

### Recombinant Sensor Cells

The present disclosure relates to recombinant sensor cells, and cell lines comprising a plurality of the recombinant sensor cells. In some embodiments, the recombinant sensor cell is capable of sensing at least two triggering events, for example molecules present in the tumor microenvironment, via recombinant proteins expressed by the cell that bind a ligand or other signal that results from the presence/occurrence of the triggering event. Once the cell senses both (or more) of the triggering events, an expressible sequence is expressed by the cell. In some embodiments, the expressible sequence encodes a reporter (e.g., mRNA or protein). Thus, presence of the at least two triggering events results in a signal from the cell, indicating the presence of the events.

In non-limiting examples, the conditions to be sensed may be pH and presence of a particular cytokine; pH and hypoxia (e.g., presence of an activated HIF protein); hypoxia and presence of a particular cytokine; etc. Conditions to be sensed may be any combination of two (or more) conditions as disclosed herein. See Table 1 for a non-limiting list of example conditions.

In some embodiments, the recombinant sensor cell comprises:
(i) a first sensor cassette comprising a first nucleic acid sequence encoding a first protein having a first ligand binding portion and a first activator portion, wherein the first ligand binding portion is capable of binding a first ligand from a first triggering event; and
(ii) a second sensor cassette comprising a second nucleic acid sequence encoding a second protein having a second ligand binding portion and a second activator portion, wherein the second ligand binding portion is capable of binding a second ligand from a second triggering event;
(iii) a first logic cassette comprising a third nucleic acid sequence comprising a promoter sequence operably linked to an expressible sequence, wherein expression from the promoter sequence is activated by the first activator portion and the second activator portion, such that the expressible sequence is expressed only after the first and second triggering events occur.

In some embodiments, the recombinant sensor cell comprises:
(i) a first sensor cassette comprising first nucleic acid sequence comprising a first signal-inducible promoter operably linked to a first expressible sequence encoding a first activator, wherein expression of the first activator is induced by a first signal from a first triggering event;
(ii) a second sensor cassette comprising a second nucleic acid sequence comprising a second signal-inducible promoter operably linked to a second expressible sequence encoding a second activator, wherein expression of the second activator is induced by a second signal from a second triggering event; and
(iii) a first logic cassette comprising a third nucleic acid sequence comprising a promoter sequence operably linked to a third expressible sequence, wherein the promoter is responsive to the first activator and the second activator;
wherein the first activator and the second activator activate expression from the promoter sequence only when both the first activator and the second activator are expressed.

Preferably, each of the first, second and third nucleic acid sequences are recombinant nucleic acid sequences, i.e., they are not endogenous to the cell.

In some embodiments, the first activator comprises a chromatin remodeler, a histone acetyltransferase, a histone deacetylase, a kinase, a methylase, a transcription factor, or a transcription co-factor. In some embodiments, the second activator comprises a chromatin remodeler, a histone acetyltransferase, a histone deacetylase, a kinase, a methylase, a transcription factor, or a transcription co-factor.

In some embodiments, the recombinant sensor cell comprises:
(i) a first sensor cassette comprising first nucleic acid sequence comprising a first signal-inducible promoter operably linked to a first expressible sequence encoding a first protein comprising a DNA binding portion and a first linker portion, wherein expression of the first protein is induced by a first signal from a first triggering event;
(ii) a second sensor cassette comprising a second nucleic acid sequence comprising a second signal-inducible promoter operably linked to a second expressible sequence encoding a second protein comprising an activator portion and a second linker portion, wherein expression of the second protein is induced by a second signal from a second triggering event, wherein interaction between the first linker portion and the second linker portion forms a functional activator protein; and
(iii) a first logic cassette comprising a third nucleic acid sequence comprising a promoter sequence operably linked to a third expressible sequence, wherein the promoter is responsive to the functional activator protein;
wherein the first protein and the second protein activate expression from the promoter sequence only when both the first protein and the second protein are expressed, such that the expressible sequence is expressed only when both the first signal and the second signal are present.

In some embodiments, the activator portion comprises a chromatin remodeler, a histone acetyltransferase, a histone deacetylase, a kinase, a methylase, a transcription factor, or a transcription co-factor.

In some embodiments, the recombinant sensor cell comprises:
(i) a first sensor cassette comprising a first nucleic acid sequence encoding a first protein having a first receptor moiety and a first activating moiety, wherein the first receptor moiety interacts with a first signal from the first triggering event;
(ii) a second sensor cassette comprising a second nucleic acid sequence encoding a second protein having a second receptor moiety and a second activating moiety, wherein the second receptor moiety interacts with a second signal from the second triggering event; and
(iii) a first logic cassette comprising a third nucleic acid sequence comprising a promoter operably linked to an expressible sequence, wherein expression from the promoter is activated by the first activating moiety and the second activating moiety, such that the expressible sequence is expressed only after the first and second triggering events occur.

In some embodiments, the first signal results in ligand binding, phosphorylation, ubiquitination, hydrolysis, nitration, sulfhydration, acetylation, lipid modification, methylation, glycosylation, propionylation, butyrylation, succinylation, malonylation, palmitoylation, and/or crotonylation of the first receptor moiety.

In some embodiments, the second signal results in ligand binding, phosphorylation, ubiquitination, hydrolysis, nitration, sulfhydration, acetylation, lipid modification, methylation, glycosylation, propionylation, butyrylation, succinylation, malonylation, palmitoylation, and/or crotonylation of the second receptor moiety.

In some embodiments, the recombinant sensor cell further comprises at least one additional logic cassette comprising a fourth nucleic acid sequence comprising a second promoter sequence operably linked to a second expressible sequence, wherein expression from the second promoter sequence is activated by the first activator portion and the second activator portion, such that the second expressible sequence is expressed only after the first and second triggering events occur. In some embodiments, the second expressible sequence encodes a reporter. In some embodiments, the second expressible sequence encodes a therapeutic molecule, a cytotoxic pathway molecule, a pro-apoptotic protein, an immunostimulator, or an immunorepressor. In some embodiments, the expressible sequence and the second expressible sequence are different sequences. Preferably, the protein or mRNA encoded by the second expressible sequence is different from a protein or mRNA encoded by the expressible sequence.

Although many of the logic cassettes described herein are AND gates, one of skill in the art would understand that logic cassettes comprising other types of logic gates are covered by the present invention. The logic cassette may comprise any type of logic gate or combination of logic gates that are turned on or off by at least two conditions, such as and without limitation, AND, NAND, OR, NOR, XOR, XNOR gates. See, e.g., Weinberg et al, Nature Biotechnology 35, pages 453-462 (2017), and International patent application publication WO 2015/188191 to Wong et al. titled “DNA Recombinase Circuits for Logical Control of Gene Expression” filed on Jun. 8, 2015, each of which is incorporated herein by reference in its entirety.

In one embodiment, the logic cassette comprises a NAND gate. In one embodiment, the recombinant sensor cell comprises a first sensor cassette and a second sensor cassette, such that each sensor cassette is responsive (e.g., transcriptionally, translationally, or activation/repression of the activity of a protein expressed therefrom) to a different triggering event, and a logic cassette comprising a nucleic acid sequence comprising a promoter operably linked to an expressible sequence, wherein the expressible sequence is expressed in the absence of the triggering events and is repressed only when both triggering events occur. For example, the sensor cassettes may express transcriptional repressors or co-repressors in response to the triggering events, and the transcriptional repressors repress the promoter of the logic cassette; the sensor cassettes may express transcriptional activators that are repressed by the triggering events; etc. In one embodiment, more than two triggering events may be required, such that expression of the expressible sequence is repressed only when all triggering events occur.

In one embodiment, the logic cassette comprises a NOR gate. In one embodiment, the presence of either (or both) of two conditions (i.e., triggering events) will repress expression of the expressible sequence. In one embodiment, more than two triggering events may be used, such that expression of the expressible sequence is repressed when one or more of the triggering events occur.

In one embodiment, the logic cassette comprises an OR gate. In one embodiment, the presence of either (or both) of two conditions (i.e., triggering events) will activate expression of the expressible sequence. In one embodiment, more than two triggering events may be used, such that expression of the expressible sequence is activated when one or more of the triggering events occur.

In one embodiment, the logic cassette comprises an XOR gate. In one embodiment, the presence of either of two conditions (i.e., triggering events), but not both, will activate expression of the expressible sequence. In one embodiment, more than two triggering events may be used, such that expression of the expressible sequence is activated when only one, only two, etc., but not all, of the triggering events occur.

In one embodiment, the logic cassette comprises an XNOR gate. In one embodiment, the presence of either of two conditions (i.e., triggering events), but not both, will repress expression of the expressible sequence. In one embodiment, more than two triggering events may be used, such that expression of the expressible sequence is repressed when only one, only two, etc., but not all, of the triggering events occur.

In some embodiments, the recombinant sensor cell comprises one or more additional sensor cassettes, each expressing an additional protein that senses an additional triggering event. In some embodiments, the expressible sequence is only expressed when the additional triggering event(s) occur.

In some embodiments is provided a cell line comprising a plurality of recombinant sensor cells as described herein.

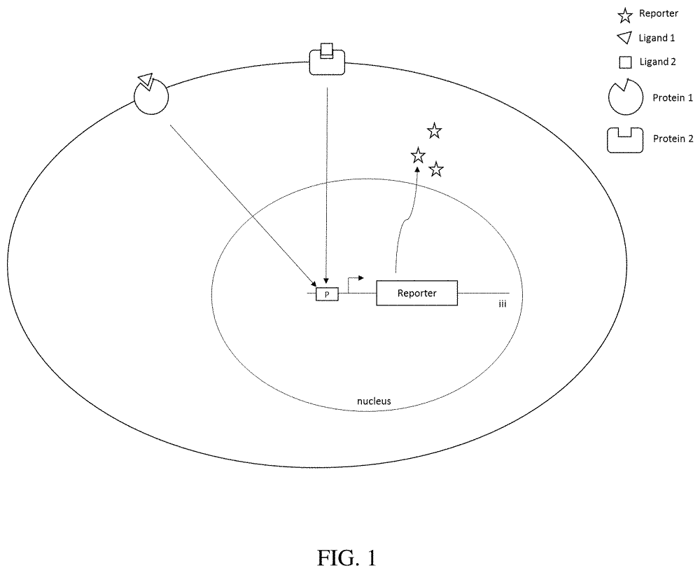

FIG. 1 is a schematic of one embodiment of a recombinant sensor cell as described herein. Protein 1 and Protein 2 are expressed from the first and second sensor cassettes, respectively (not shown). Ligand 1 is a signal from a first triggering event, and Ligand 2 is a signal from a second triggering event. Upon binding of Ligand 1 to Protein 1, Protein 1 initiates a signal that results in binding of a transcriptional activator or transcriptional co-activator (or removal of a transcriptional repressor) at the promoter (P) of the logic cassette (iii). However, transcription from P is not initiated until Ligand 2 binds to Protein 2, resulting in a second signal that results in binding of a second transcriptional activator or transcriptional co-activator (or removal of a second transcriptional repressor) at the promoter (P) of the logic cassette (iii). The logic cassette (iii) encodes a reporter gene that is expressed upon transcription from the promoter (P).

Although Protein 1 and Protein 2 are depicted in FIG. 1 as membrane-bound proteins, one of skill in the art would understand that one or both proteins may be intracellular proteins, e.g. proteins that sense a signal from a triggering event indirectly (e.g., via a signal cascade through endogenous cell protein(s) that are activated by the triggering event) or directly (e.g., by binding a ligand from the signaling event, wherein the ligand is capable of active or passive diffusion into the cell).

For example, and without limitation, a recombinant sensor cell as described herein may comprise a first sensor cassette comprising a gene encoding an IL-8 receptor (e.g., CXCR1 or CXCR2) operatively linked to a constitutive promoter, a second sensor cassette comprising a gene encoding hypoxia inducible factor alpha (HIFa) operatively linked to a constitutive promoter, and a first logic cassette comprising a promoter that is activated only when activated HIFa and a down-stream effector of the IL-8 receptor (e.g., a transcription factor activated by the IL-8 pathway). When the recombinant sensor cell is in an environment where both IL-8 and hypoxia are present, e.g., a tumor microenvironment, the reporter gene is expressed. In contrast, when only one of hypoxia or IL-8 is present, e.g., hypoxia in the muscle, the reporter gene is not expressed. In some embodiments, a therapeutic molecule, e.g., IL-15, is expressed from the logic cassette instead of or in addition to a reporter.

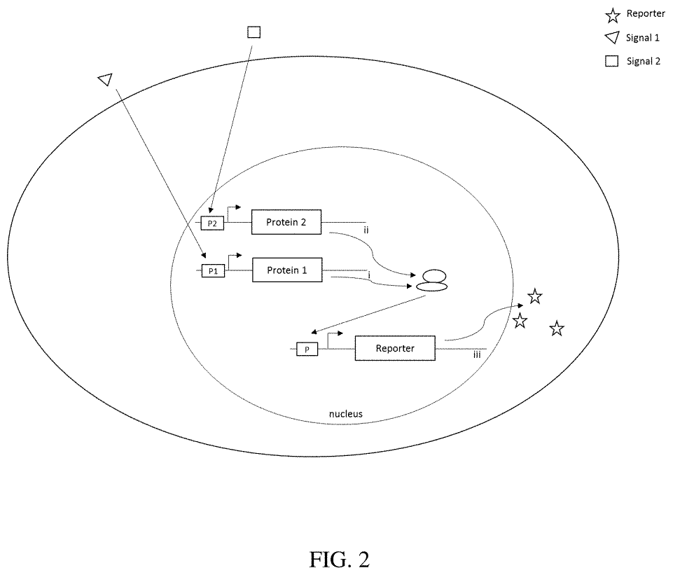

FIG. 2 is a schematic of another embodiment of a recombinant sensor cell as described herein. The presence of Ligand 1 results in activation of expression of Protein 1 from promoter 1 (P1) in the first sensor cassette (i). The presence of Ligand 2 results in activation of expression of Protein 2 from promoter 2 (P2) in the second sensor cassette (ii). Protein 1 and Protein 2 interact, e.g., via linkers on each protein (e.g., leucine zipper, nanobody/peptide, streptavidin/strep-tag, or an affinity clamp), to form a single activator protein that activated transcription from the logic cassette (iii).

### Sensed Environments

The sensed environment may be any environment of interest, e.g. to a clinician. In some embodiments, the first triggering event and/or the second triggering event is present in a tumor cell microenvironment. In some embodiments, the first triggering event and/or the second triggering event is present in a microenvironment characterized by the presence of fatty tissue or acne. In some embodiments, the first triggering event and/or the second triggering event is present in (e.g., characteristic of) a particular tissue, for example and without limitation prostate, liver, lung, breast, brain, skin, blood, hair follicles, heart, bladder, uterus, cervix, ovary, colon, etc. In some embodiments, the sensed environment is an inflamed tissue. In some embodiments, the sensed environment is a healthy tissue. In some embodiments, the sensed environment is an infected tissue.

In some embodiments, the first triggering event is selected from cell density, cell stress, pH, hypoxia, heat, presence of a molecule of interest, or concentration of a molecule of interest. In some embodiments, the second triggering event is selected from cell density, cell stress, pH, hypoxia, heat, presence of a molecule of interest, or concentration of a molecule of interest. In some embodiments, the first triggering event or the second triggering event is the presence of intracellular components (histones, ribosomal proteins, necrosis proteins, etc.) in the intercellular/extracellular environment. In some embodiments, the first or second triggering event is an event that stimulates growth of a cell (e.g., vascularization, presence of VEGF, etc.). In some embodiments, the first or second triggering event is an event that indicates apoptosis, cell stress, necrosis, or loss of cell adhesion.

In some embodiments, the molecule of interest is a cytokine, a chemokine, a metabolite, an exosome, an enzyme, a sugar, an intracellular component, a soluble checkpoint inhibitor, a signaling factor, a pathogen (e.g., a virus, a yeast cell, or a bacterial cell).

### Triggering Events/Conditions and Recombinant Proteins

Molecules that indicate various triggering events are known in the art, as are proteins that sense those molecules. Non-limiting examples are provided in Table 1.

| Triggering Event | Triggering Molecule(s) | Sensor Protein(s) | References* |
|---|---|---|---|
| cell density | other cells in proximity | Hippo pathway, YAP, | Sharif et al., Future |
| | | TAZ, GPCRs, e-cadherin | Oncol. 2015 December; |
| | | | 11(24): 3253-3260. |
| cytokines | cytokine (e.g., chemokines, | cytokine receptors | Bagley et al., Blood |
| | interferons, interleukins, | | 1997 89: 1471-1482. |
| | lymphokines, and tumour | | |
| | necrosis factors) | | |
| chemokines | chemokines (e.g., CXC, | chemokine receptors (e.g., | Palomino and Marti, |
| | CX3C, CC, or C chemokines) | CXC chemokine receptors, | Einstein (Sao Paulo) |
| | | CC chemokine receptors, | 2015 July-September; |
| | | CX3C chemokine receptors | 13(3): 469-473. |
| | | and XC chemokine | |
| | | receptors) | |
| pH | proton (H+) | acid-sensing ion channels | Damaghi et al., Front. |
| | | (ASICs) and proton-sensing | Physiol. 2013 Vol. 4, |
| | | GPCRs (e.g., GPR4, | Article 370. |
| | | TDAG8, and OGR1) | |
| metabolites | metabolite (e.g., alcohol, | receptors (e.g., GPCRs) | Blad et al., Nature |
| | amino acids, nucleotides, | | Reviews Drug |
| | antioxidants, organic acids, | | Discovery volume 11, |
| | polyols, vitamins, fatty acids, | | pages 603-619 (2012); |
| | saccharides, lactate, ketone) | | Yuan et al., Mol Cell. |
| | | | 2013 Feb. 7; 49(3): 379-387. |
| exosomes and other | exosomes, microvesicles, | receptors | Tkach and Thery, Cell |
| extracellular | ectosomes, microparticles, | | 164, Mar. 10, 2016, |
| vesicles | surface molecules | | 1226-1232. |
| sugars | sugar or sugar moiety | lectins, sucrose | Ghazarian, et al., Acta |
| | | transporters, glucosensors | Histochem. 2011 May; |
| | | | 113(3): 236-247. |
| Soluble immune | PD-L1 | PD1, CTLA4 | Manson et al., Annals of |
| checkpoint markers | | | Oncology, Volume 27, |
| | | | Issue 7, 1 Jul. 2016, |
| | | | Pages 1199-1206. |
| angiogenic factors | VEGF, bFGF, TGF-α, TGF- | receptors for each factor | Nishida et al., Vasc |
| | β, platelet-derived endothelial | (e.g., VEGFR, TGFR, | Health Risk Manag. |
| | growth factor, granulocyte | EGFR, IL receptors, etc.) | 2006 September; |
| | colony-stimulating factor, | | 2(3): 213-219. |
| | placental growth factor, | | |
| | interleukin-8, hepatocyte | | |
| | growth factor, epidermal | | |
| | growth factor | | |
| tumor-specific | neoepitopes; tumor- | antibodies, CARs | WO2017205810; |
| factors | associated antigens | | WO2017066256; |
| | | | WO2018089637 |
| apoptosis | apoptosis-associated factors | receptors, antibodies | Ward et al., British |
| | (e.g., cleaved cytokeratin-18 | | Journal of Cancer |
| | (c-CK18), cleaved caspase-3 | | volume 99, pages 841-846 |
| | (c-cas-3), cleaved lamin A (c- | | (16 Sep. 2008) |
| | lam-A), phosphorylated | | |
| | histone H2AX | | |
| | (gammaH2AX), cleaved | | |
| | poly(ADP ribose) polymerase | | |
| | (c-PARP), | | |
| | phosphatidylserine, | | |
| | Cytokeratins, Nucleosomal | | |
| | DNA, Apo-1/Fas, Fas ligand | | |
| | (sFasL), Bcl-2/Bcl-xl/Mcl-1, | | |
| | p53, phospo-p53, p21wafi, | | |
| | pH2AX, cytochrome c, | | |
| | Activated caspases 2, 3, 7, 8 | | |
| | and 9, fortilin) | | |
| stress | stress proteins, cytokines, | receptors, etc. | Milisav (2011). Cellular |
| | chemokines, apoptotic | | Stress Responses, Adv. |
| | factors, etc. | | in Regen. Med., S. |
| | | | Wislet-Gendebien (Ed.) |
| loss of adhesion | cell adhesion molecules | CAMs | |
| | (CAMs, e.g., integrins, | | |
| | immunoglobulin (Ig) | | |
| | superfamily, cadherins, and | | |
| | selectins) | | |
| pathogens | pathogen (bacterial cell, | receptors (e.g., TLRs), | Akira et al. Cell |
| | fungus, virus, parasite, etc) or | antibodies, lectins, | 124, 783-801, |
| | an antigen or toxin therefrom | | Feb. 24, 2006. |
| hypoxia | intracellular oxygen levels | HIFα, HIHβ, heme protein, | Zhu and Bunn, Science. |
| | | prolyl hydroxylase | 2001 Apr. 20; |
| | | | 292(5516): 449-151. |
| heat/temperature | heat | vanilloid receptor | U.S. Pat. No. 7,396,910; Moqrich |
| | | | et al., Science |
| | | | 4 Mar. 2005: |
| | | | Vol. 307, Issue |
| | | | 5714, pp. 1468-1472 |

*Each reference is incorporated herein by reference in its entirety.

In some embodiments, the proteins that sense a triggering molecule and/or activate expression are fusion proteins, for example having a sensor (e.g., ligand-biding) domain from one protein and an effector domain (e.g., activator domain or repressor domain) from a different protein. In some embodiments, the same protein senses (e.g., binds) the trigger molecule and activates or represses transcription, e.g., of the expressible sequence. In some embodiments, the sensor protein modulates activity of another protein that in turn modulates transcription, e.g., of the expressible sequence. The protein that is modulated by the sensor protein may be an endogenous protein, or it may be a recombinant protein.

In some embodiments, the sensor protein is a chimeric antigen receptor (CAR) or similar to a CAR. That is, the ligand-binding domain of the sensor comprises an antibody (or fragment thereof) to the trigger molecule, and the effector domain comprises a signaling molecule that results in repression or activation of the expressible sequence upon binding of the trigger molecule to the antibody (or fragment thereof).

In some embodiments, the triggering event is irradiation of a tumor. Irradiation of a tumor causes bystander effects, where non-irradiated cells near the irradiated cells are affected by signals from the irradiated tumor. Irradiation of a tumor can also cause abscopal effects, i.e., effects in a site that is distant from the tumor. The triggering molecule may be a molecule that results from irradiation or bystander effect, for example and without limitation, cytokines (e.g., IL-1, IL-2, IL-6, IL-8, TNFα, TGFβ), cyclooxygenase-2, miRNA (e.g., miR-29, miR-16, miR-17, MIR-29a and MIR-29b), siRNA and piRNA. See, J Biomed Phys Eng. 2014 December; 4(4): 163-172, which is incorporated herein by reference in its entirety.

Of course, it should be recognized that a triggering molecule may be a molecule as indicated above, or may be a down-stream effector of such a molecule.

### Expressible Sequences

In some embodiments, the expressible sequence encodes a reporter. In some embodiments, the reporter may be any reporter protein or RNA that can be detected in vivo, ex vivo, or in vitro. In some embodiments, the reporter is selected from a fluorescent protein, a cell surface marker, a detectable RNA molecule, a detectable DNA molecule, a luciferase, or a reporter enzyme. Such reporters are well known in the art. In some embodiments, the reporter can be detected by imaging the cell (e.g., within a patient or in vitro). In some embodiments, the reporter can be detected in a biological sample. In some embodiments, the reporter can be detected in a liquid biopsy (e.g., blood sample), biopsy, urine, fecal sample, mucous, or other bodily fluid.

In some embodiments, the expressible sequence encodes a binding peptide linked to a reporter. For example, an antibody, ligand for a protein of interest (e.g., a cell surface protein expressed by a tumor cell), or other binding peptide may be linked to (fused with) a reporter such as a fluorescent protein. In some embodiments, the binding peptide recognizes (binds to) an epitope that is present in a particular microenvironment (e.g., tumor microenvironment). In some embodiments, expression of the binding peptide linked to a reporter allows visualization of a particular microenvironment or cell. This may be used, for example, to visualize boundaries of a tumor, presence of metastases, individual cancer cells in a sample, and the like. In some embodiments, the antibody is a single domain antibody (e.g., camelid antibody) or epitope-binding fragment thereof. Single domain antibodies are known in the art, for example as described in Harmsen and Haard, Appl Microbiol Biotechnol. 2007 November; 77(1): 13-22, which is incorporated herein by reference in its entirety.

In some embodiments, the expressible sequence encodes a therapeutic molecule, a cytotoxic pathway molecule, a pro-apoptotic protein, an immunostimulator, or an immunorepressor. In some embodiments, the expressible sequence encodes a perforin or granzyme. In some embodiments, the expressible sequence encodes a pro-inflammatory cytokine (e.g., IL-8, TGF-β) or other molecule to reduce immune suppression. In some embodiments, the expressible sequence encodes pro-apoptotic protein(s). In some embodiments, the expressible sequence encodes a cell surface marker to allow isolation of the cell from a sample, e.g. a biological sample, e.g., blood.

### Vectors

Any type of vector may be used, including, without limitation, viruses, plasmids, and the like. In some embodiments, the nucleic acids (e.g., the first, second and/or third nucleic acids) are present on one or more vectors in the cell. In some embodiments, one, two, three or more of the nucleic acids is present in a single vector.

In some embodiments is provided a recombinant vector comprising one or more of:
(i) a first sensor cassette comprising a first nucleic acid sequence encoding a protein having a first receptor moiety and a first activating moiety, wherein the first receptor moiety binds a first signal from the first triggering event;
(ii) a second sensor cassette comprising a second nucleic acid sequence encoding a protein having a second receptor moiety and a second activating moiety, wherein the second receptor moiety binds a second signal from the second triggering event; and
(iii) a first logic cassette comprising a third nucleic acid sequence comprising a promoter operably linked to an expressible sequence, wherein expression from the promoter is activated by the first activating moiety and the second activating moiety, such that the expressible sequence is expressed only after the first and second triggering events occur.

### Type of Cells

In some embodiments, the recombinant sensor cell is an immune cell, a stem cell, a bacterial cell, or a parasite. In some embodiments, the immune cell is an immunocompetent cell. In some embodiments, the immune cell is a natural killer cell, a B cell, or a T cell. In some embodiments, the cell is derived from a subject, e.g., a patient to be treated.

In some embodiments, the bacterial cell is Escherichia coli. In some embodiments, the bacterial cell does not trigger the endotoxic response in mammalian cells. In some embodiments, the bacterial cell is a ClearColi® cell (Lucigen®, Madison, Wis.).

In some embodiments, the parasite is a nematode, a spirochete, or a fungus.

In some embodiments, the recombinant sensor cell is an enucleated cell that is capable of transcription and translation. In some embodiments, the recombinant sensor cell is an exosome that is capable of transcription and translation.

The present disclosure also relates, in part, to methods of making recombinant sensor cells as described herein. Methods of inserting recombinant nucleic acids into cells are well known in the art. See, e.g., M. R. Green and J. Sambrook, Molecular Cloning: A Laboratory Manual (Fourth Edition), Cold Spring Harbor Laboratory Press; (Jun. 15, 2012).

In some embodiments is provided a method for producing a recombinant therapeutic cell as described herein, the method comprising introducing the first logic cassette, first sensor cassette, and second sensor cassette into the cell.

In some embodiments, the nucleic acids are transiently transfected into the cell. In some embodiments, the nucleic acids are stably transfected into the cell.

### Methods of Using Recombinant Sensor Cells

### In Vivo Methods

A recombinant cell as described herein may be administered to a subject. As would be apparent to a person of skill in the art, the mode of administration may differ, depending on the disease to be treated, the subject, the area or tissue to be treated, etc. In some embodiments is provided a composition comprising a recombinant sensor cell as described herein which is formulated for topical, intravenous, oral, nasal, vaginal, or anal delivery.

In some embodiments is provided a topical composition comprising a recombinant sensor cell as described herein. In some embodiments is provided an injectable composition comprising a recombinant sensor cell as described herein. In some embodiments, the injectable composition is formulated for intravenous administration. In some embodiments is provided an oral composition comprising a recombinant sensor cell as described herein.

In some embodiments is provided a method of detecting at least two triggering events in a tumor microenvironment, the method comprising contacting the tumor microenvironment with a recombinant sensor cell as described herein, and determining the presence or absence of an RNA or polypeptide expressed from the expressible sequence.

In some embodiments is provided a method of treating a tumor, the method comprising contacting the tumor microenvironment with a recombinant sensor cell as described herein, wherein the expressible sequence encodes a molecule that treats the tumor. In some embodiments, the molecule that treats the tumor is an immune stimulating cytokine, a chemokine, a pro-apoptotic protein, an antisense RNA, a chimeric antigen receptor, or a therapeutic antibody.

In some embodiments, the tumor is in a subject and the recombinant sensor cell is derived from the subject.

By way of example only, and without limitation, a recombinant sensor cell as described herein may be used to detect a tumor in a patient by sensing at least two triggering events in a tumor microenvironment. Upon sensing the events, a reporter is expressed that can be detected by a clinician. In some embodiments, a plurality of recombinant sensor cells can be administered to the patient to map the tumor microenvironment based on the triggering events. The recombinant sensor cell similarly can be used to detect metastases by expressing the reporter when the cell is in a tumor microenvironment produced by the metastasis. A topical formulation comprising the recombinant sensor cell could be used, for example, to indicate the presence of melanoma on the skin of a patient.

By way of another non-limiting example, a recombinant sensor cell as described herein can detect whether a tumor is likely to be responsive to a particular anti-cancer treatment by determining whether one or more particular conditions is present in the tumor microenvironment (e.g., presence of VEGF for treatment with bevacizumab; high tumor HLA expression for treatment with checkpoint inhibitors; presence of particular tumor-associated antigens or neoepitopes for treatment with a variety of therapeutics).

### In Vitro Methods

In some embodiments is provided a method of detecting at least two triggering events in a biological sample, the method comprising contacting the biological sample with a recombinant sensor cell as described herein, and determining the presence or absence of an RNA or polypeptide expressed from the expressible sequence.

In some embodiments, the biological sample is from a subject having or suspected of having cancer. In some embodiments, the biological sample comprises blood, urine, a biopsy, saliva, hair, skin, or feces. In some embodiments, the biological sample comprises cell free DNA and/or cell free RNA. In some embodiments, the biological sample comprises a tumor sample (e.g., formalin fixed paraffin embedded (FFPE) patient tissue) on a microscope slide.

By way of example only, and without limitation, a recombinant sensor cell as described herein may be used to detect a tumor in a biopsy, blood sample, or other biological sample. In one embodiment, the recombinant sensor cell as described herein may be used to detect regions where cancer is found in a biopsy or other tumor sample (e.g., on a microscope slide). In one embodiment, the presence of cancer cells on the slide is used to determine the mask for laser microdissection from the slide.

By way of another non-limiting example, a recombinant sensor cell as described herein can be administered orally to a patient, such that samples (e.g., urine or fecal samples) are analyzed to detect the presence of the reporter.

In some embodiments, the recombinant sensor cells are used to detect a cancer using a reporter that can be viewed by eye under certain conditions (e.g., a fluorescent reporter). In some embodiments, the cancer is a skin cancer. In some embodiments, the skin cancer or suspected skin cancer is contacted with recombinant sensor cells as described herein. The recombinant sensor cells sense the skin cancer microenvironment (e.g., presence of matrix metalloproteinases, cytokines, CFI complement factor I, CFH complement factor H, FHL-1 Factor H-like protein 1, growth factors, angiogenic factors, hypoxia, etc.). The sensor cells then express the expressible sequence that is detectable by eye, and the clinician can ascertain whether cancer cells are present, the borders of the cancer, etc.

In some embodiments, the recombinant sensor cells are used to detect a cancer using a reporter that can be detected by medical imaging (e.g., X-ray radiography, magnetic resonance imaging, medical ultrasonography/ultrasound, endoscopy, elastography, tactile imaging, thermography, medical photography, nuclear medicine functional imaging techniques, e.g., positron emission tomography (PET) and Single-photon emission computed tomography (SPECT), CT etc.). For example, the recombinant sensor cells can be injected into a patient (intravenous, intraperitoneal, at the site of a tumor or suspected tumor, etc.), followed by medical imaging to locate the region of the patient where the expressible sequence (e.g., reporter) is expressed.

In some embodiments, the recombinant sensor cells are used to detect a cancer, such as esophageal cancer, stomach cancer, throat cancer, intestinal cancer, bladder cancer, or colon cancer, wherein the cells are administered orally to the patient, the cells express the reporter in the presence of the cancer, and the reporter is excreted by the patient (e.g., in the urine or feces). The excretion is assayed for presence of the reporter.

In some embodiments, the recombinant sensor cells are used to detect metastases of a tumor. In some embodiments, the recombinant sensor cells are used to determine the effectiveness of a therapy and/or monitor progress of a therapy. For example, the recombinant sensor cells may be administered one or more times to a patient undergoing an anti-cancer therapy to ascertain the size and/or location of a tumor, location of metastases, etc., and to monitor whether the tumor size changes, more or less metastases are detectable, etc., during or after treatment with the anti-cancer therapy. The recombinant sensor cells also may be used to determine whether a tumor microenvironment changes in the patient over time, e.g. as a result of treatment.

### Kits

In some embodiments is provided a kit comprising at least one of:
(i) a first sensor cassette comprising a first nucleic acid sequence encoding a first protein having a first ligand binding portion and a first activator portion, wherein the first ligand binding portion is capable of binding a first signal from the first triggering event; and
(ii) a second sensor cassette comprising a second nucleic acid sequence encoding a second protein having a second ligand binding portion and a second activator portion, wherein the second ligand binding portion is capable of binding a second signal from the second triggering event;
(iii) a first logic cassette comprising a third nucleic acid sequence comprising a promoter sequence operably linked to an expressible sequence, wherein expression from the promoter sequence is activated by the first activator portion and the second activator portion, such that the expressible sequence is expressed only after the first and second triggering events occur;
such that when both the first signal and the second signal are present the expressible sequence is expressed.

In some embodiments is provided a kit comprising at least one of:
(i) a first sensor cassette comprising first nucleic acid sequence comprising a first signal-inducible promoter operably linked to a first expressible sequence encoding a first activator, wherein expression of the first activator agent is induced by a first signal from the first triggering event;
(ii) a second sensor cassette comprising a second nucleic acid sequence comprising a second signal-inducible promoter operably linked to a second expressible sequence encoding a second activator, wherein expression of the second activator agent is induced by a second signal from the second triggering event; and
(iii) a first logic cassette comprising a third nucleic acid sequence comprising a promoter sequence operably linked to a third expressible sequence, wherein the promoter is responsive to the first activator and the second activator;
wherein the first activator and the second activator activate expression from the promoter sequence only when both the first activator and the second activator are present, such that the expressible sequence is expressed only when both the first signal and the second signal are present.

In some embodiments is provided a kit comprising at least one of:
(i) a first sensor cassette comprising a first nucleic acid sequence encoding a protein having a first receptor moiety and a first activating moiety, wherein the first receptor moiety binds a first signal from the first triggering event;
(ii) a second sensor cassette comprising a second nucleic acid sequence encoding a protein having a second receptor moiety and a second activating moiety, wherein the second receptor moiety binds a second signal from the second triggering event; and
(iii) a first logic cassette comprising a third nucleic acid sequence comprising a promoter operably linked to an expressible sequence, wherein expression from the promoter is activated by the first activating moiety and the second activating moiety, such that the expressible sequence is expressed only after the first and second triggering events occur.

In some embodiments, the kit comprises at least two of the first sensor cassette, second sensor cassette, and/or the first logic cassette.

In some embodiments, the first sensor cassette, second sensor cassette, and the first logic cassette are present in a single vector. In some embodiments, the first sensor cassette, second sensor cassette, and/or the first logic cassette are present in two or more separate vectors.

In some embodiments, the kit further comprises a cell (or plurality of cells) to be transfected/infected with the nucleic acid sequences/vector(s).

What is claimed is:

1. A recombinant sensor cell comprising:
(i) a first sensor cassette comprising a first recombinant nucleic acid sequence encoding a first protein having a first ligand binding portion and a first activator portion, wherein the first ligand binding portion is capable of binding a first ligand from a first triggering event; and
(ii) a second sensor cassette comprising a second recombinant nucleic acid sequence encoding a second protein having a second ligand binding portion and a second activator portion, wherein the second ligand binding portion is capable of binding a second ligand from a second triggering event;
(iii) a first logic cassette comprising a third recombinant nucleic acid sequence comprising a promoter sequence operably linked to an expressible sequence, wherein expression from the promoter sequence is activated by the first activator portion and the second activator portion, such that the expressible sequence is expressed only after the first and second triggering events occur.

2. A recombinant sensor cell comprising:
(i) a first sensor cassette comprising first recombinant nucleic acid sequence comprising a first signal-inducible promoter operably linked to a first expressible sequence encoding a first activator, wherein expression of the first activator is induced by a first signal from a first triggering event;
(ii) a second sensor cassette comprising a second recombinant nucleic acid sequence comprising a second signal-inducible promoter operably linked to a second expressible sequence encoding a second activator, wherein expression of the second activator is induced by a second signal from a second triggering event; and
(iii) a first logic cassette comprising a third recombinant nucleic acid sequence comprising a promoter sequence operably linked to a third expressible sequence, wherein the promoter is responsive to the first activator and the second activator;
wherein the first activator and the second activator activate expression from the promoter sequence only when both the first activator and the second activator are expressed.

3. The recombinant sensor cell of claim 1, wherein the first activator comprises a chromatin remodeler, a histone acetyltransferase, a histone deacetylase, a kinase, a methylase, a transcription factor, or a transcription co-factor.

4. The recombinant sensor cell of claim 1, wherein the second activator comprises a chromatin remodeler, a histone acetyltransferase, a histone deacetylase, a kinase, a methylase, a transcription factor, or a transcription co-factor.

5. The recombinant sensor cell of claim 1, wherein the first triggering event and the second triggering event are independently selected from cell density, pH, hypoxia, radio signal, MRI, heat, presence of a molecule of interest, or concentration of a molecule of interest.

6. The recombinant sensor cell of claim 5, wherein the molecule of interest is a cytokine, a chemokine, a metabolite, an exosome, an enzyme, a sugar, an intracellular component, a soluble checkpoint inhibitor, a signaling factor, a virus, a yeast cell, or a bacterial cell.

7. The recombinant sensor cell of claim 1, wherein the first triggering event and/or the second triggering event is present in a tumor cell microenvironment.

8. The recombinant sensor cell of claim 1, wherein the expressible sequence encodes a reporter.

9. The recombinant sensor cell of claim 1, wherein the expressible sequence encodes a therapeutic molecule, a cytotoxic pathway molecule, a pro-apoptotic protein, an immuno stimulator, or an immunorepressor.

10. The recombinant sensor cell of claim 1, wherein the recombinant sensor cell is an immune cell, a stem cell, a bacterial cell, or a parasite.

11. The recombinant sensor cell of claim 1, wherein the cell is derived from a patient.

12. The recombinant sensor cell of claim 1, further comprising at least one additional logic cassette comprising a fourth nucleic acid sequence comprising a second promoter sequence operably linked to a second expressible sequence, wherein expression from the second promoter sequence is activated by the first activator portion and the second activator portion, such that the second expressible sequence is expressed only after the first and second triggering events occur.

13. A cell line comprising a plurality of the recombinant sensor cell of claim 1.

14. A topical composition comprising a recombinant sensor cell of claim 1.

15. A composition comprising a recombinant sensor cell of claim 1 which is formulated for oral delivery.

16. A method for producing a cell of claim 1, the method comprising introducing the first logic cassette, first sensor cassette, and second sensor cassette into the cell.

17. A method of detecting at least two triggering events in a tumor microenvironment, the method comprising contacting the tumor microenvironment with a recombinant sensor cell of claim 1 and determining the presence or absence of an RNA or polypeptide expressed from the expressible sequence.

18. A method of treating a tumor, the method comprising contacting the tumor microenvironment with a recombinant sensor cell of claim 1, wherein the expressible sequence encodes a molecule that treats the tumor.

19. A recombinant vector comprising:
(i) a first sensor cassette comprising a first nucleic acid sequence encoding a protein having a first receptor moiety and a first activating moiety, wherein the first receptor moiety binds a first signal from the first triggering event;
(ii) a second sensor cassette comprising a second nucleic acid sequence encoding a protein having a second receptor moiety and a second activating moiety, wherein the second receptor moiety binds a second signal from the second triggering event; and
(iii) a first logic cassette comprising a third nucleic acid sequence comprising a promoter operably linked to an expressible sequence, wherein expression from the promoter is activated by the first activating moiety and the second activating moiety, such that the expressible sequence is expressed only after the first and second triggering events occur.

20. A kit comprising:
(i) a first sensor cassette comprising a first nucleic acid sequence encoding a first protein having a first ligand binding portion and a first activator portion, wherein the first ligand binding portion is capable of binding a first signal from the first triggering event; and
(ii) a second sensor cassette comprising a second nucleic acid sequence encoding a second protein having a second ligand binding portion and a second activator portion, wherein the second ligand binding portion is capable of binding a second signal from the second triggering event;
(iii) a first logic cassette comprising a third nucleic acid sequence comprising a promoter sequence operably linked to an expressible sequence, wherein expression from the promoter sequence is activated by the first activator portion and the second activator portion, such that the expressible sequence is expressed only after the first and second triggering events occur;
such that when both the first signal and the second signal are present the expressible sequence is expressed.
